(19) United States
(12) Patent Application Publication
(10) Pub. No.: US 2018/0353489 A1
(43) Pub. Date: Dec. 13, 2018

(54) Antibacterial Pharmaceutical Combination and Method for Treating Gram-Negative Bacteria Infections

(71) Applicant: TENNOR THERAPEUTICS (SUZHOU) LIMITED, Suzhou (CN)

(72) Inventors: Zhenkun Ma, Suzhou (CN); Gregory Robertson, Suzhou (CN); Xiaomei Wang, Suzhou (CN)

(21) Appl. No.: 16/108,146

(22) Filed: Aug. 22, 2018

Related U.S. Application Data
(63) Continuation of application No. PCT/CN2017/078752, filed on Mar. 30, 2017.

(30) Foreign Application Priority Data
Apr. 18, 2016 (CN) 201610238915.5

(51) Int. Cl. A61K 31/4375; A61P 31/04; A61K 38/12

(52) U.S. Cl. CPC A61K 31/4375; A61K 38/12; A61P 31/04

(57) ABSTRACT

Provide herein is an antibacterial pharmaceutical combination for treating Gram negative bacterial infections, including a compound TNP-2092 and a cell membrane permeabilizer. Also provided herein is a method for treating Gram-negative bacteria infections in a subject, which includes administrating to the subject the antibacterial pharmaceutical combination.

### CROSS-REFERENCE TO RELATED APPLICATIONS

The present application is a continuation application of International (PCT) Application No. PCT/CN2017/078752, filed on Mar. 30, 2017, which claims foreign priority of Chinese Patent Application No. 201610238915.5 filed on Apr. 18, 2016. The entire disclosure and contents of the above applications are hereby incorporated by reference herein.

### TECHNICAL FIELD

The present disclosure relates to the pharmaceutical field, in particular to an antibacterial pharmaceutical combination and a method for treating Gram-negative bacteria infections.

### BACKGROUND

Due to the development of antibiotic resistance, the treatment of Gram-negative bacteria infections is facing significant challenges. Clinically important Gram-negative pathogen include: Escherichia coli, Klebsiella pneumoniae, Acinetobacter baumannii, Pseudomonas aeruginosa, Stenotrophomonas maltophilia, Salmonella typhi, non-typhoidal Salmonella, Shigella and so on. Klebsiella pneumoniae is a major pathogen in common hospital-acquired infections, and carbapenems have been the last defense against the infection of this bacterium; however, more than half of Klebsiella pneumoniae infections have been caused by strains resistance to carbapenems in some areas, and the resistance has spread around the world. In China, Carbapenem-Resistant Enterobacteriaceae (CRE) are increasing. Klebsiella pneumoniae is the most common bacterium in enterobacteriaceae, and the resistance rates of carbapenem-resistant Klebsiella pneumoniae from 2009 to 2012 are 2.1%, 6.2%, 9.3% and 10.8%, respectively, according to CHINET.

Investigation employing derivatives of the Escherichia coli strain D21 bearing specific rifamycin- and/or quinolone-resistance mutations combined with lpxC and tolC mutant alleles (both alone and in combination) revealed that the antimicrobial activity of TNP-2092 is impacted by: (1) basal and/or inducible efflux mechanism(s) that utilize tolC as an outer membrane channel; (2) improved intracellular access as afforded by the lpxC mutation. Based on these results, it is hypothesized that the overall antimicrobial activity of TNP-2092 versus Gram-negative bacteria might be improved if co-administered with a suitable antibiotic potentiating agent that either improves TNP-2092 penetration or uptake into bacteria, or blocks antimicrobial efflux.

### SUMMARY

In one aspect, the present disclosure provides an antibacterial pharmaceutical combination for treating Gram-negative bacteria infections, including a compound TNP-2092 and a cell membrane permeabilizer,

the chemical name of TNP-2092 is:

(R)-3-[[[4-[1-[1-(3-carboxyl-1-cyclopropyl-7-fluorine-9-methyl-4-oxygen-4-hydrogen-8-quinolizinyl)-3-pyrrolidinyl]cyclopropyl](methyl)amino]-1-piperidyl]imidogen]methyl]-rifamycin SV, with a structural formula as follows:

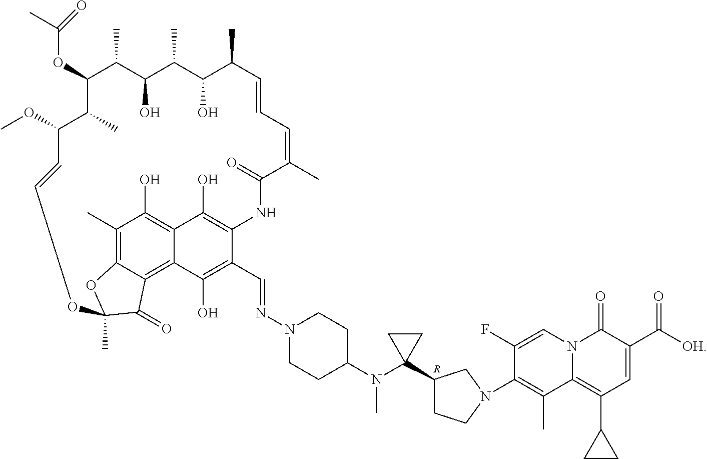

In some embodiments, the combination includes TNP-2092 and a cell membrane permeabilizer with a weight ratio of 3:400-125:4.

In some embodiments, the cell membrane permeabilizer is polymyxin B or polymyxin E.

In some embodiments, the combination includes TNP-2092 and polymyxin B with a weight ratio of 3:400-125:4.

In some embodiments, the combination includes TNP-2092 and polymyxin E with a weight ratio of 3:400-25:3.

In another aspect, a method for treating Gram-negative bacteria infections in a subject is provided, which includes administrating to the subject the antibacterial pharmaceutical combination described above.

In some embodiments, the compound TNP-2092 and the cell membrane permeabilizer in the antibacterial pharmaceutical combination are administrated separately, or in a form of a mixture.

In some embodiments, the Gram-negative bacteria includes at least one selected from the group consisting of Escherichia coli, Klebsiella pneumoniae, Acinetobacter baumannii, Pseudomonas aeruginosa, Stenotrophomonas maltophilia, Salmonella typhi, non-typhoidal Salmonella and Shigella.

Compared with prior discovery, the present disclosure has the following advantages: the antibacterial pharmaceutical combination for treating Gram-negative bacteria infections in the present disclosure, which utilizes the combination of TNP-2092 and a cell membrane permeabilizer, has stronger antibacterial activity than that when TNP-2092 or the cell membrane permeabilizer is used alone; has a synergistic antibacterial effect, and can be used for treating Gram-negative bacteria infections, including drug-resistant bacteria infection.

### DESCRIPTION OF DRAWINGS

FIG. 1 is the test result of Minimum Bactericidal Concentration (MBC) of TNP-2092 and polymyxin B pharmaceutical combination against Escherichia coli ATCC 25922 strain by checkerboard method;

FIG. 2 is the test result of Minimum Bactericidal Concentration (MBC) of TNP-2092 and polymyxin E pharmaceutical combination against Escherichia coli ATCC 25922 strain by checkerboard method.

### DETAILED DESCRIPTION

To further understand the technical features, purpose and advantages of the present disclosure, the technical details of the present disclosure are described below. The examples below should not be interpreted as the limitation of the scope of the present disclosure.

A standard 96-well plate checkerboard test platform for the determination of Minimum Inhibitory Concentration (MIC) and Minimum Bactericidal Concentration (MBC) as endpoints is used for the test of the antibacterial activity of the pharmaceutical combination in vitro. Escherichia coli ATCC 25922 strain is used as the representative strain of Gram-negative bacteria in initial investigations. The correlation between the observed results and the major Gram-negative bacteria in hospital—Pseudomonas aeruginosa, Klebsiella pneumoniae, Acinetobacter baumannii and Stenotrophomonas maltophilia—is assessed by a similar method.

Escherichia coli ATCC 25922 is a quality control strain for MIC testing as recommended by the Clinical and Laboratory Standards Institute (CLSI), which was obtained originally from the American Type Culture Collection (ATCC) repository and was used as a model of Gram-negative pathogen described herein. Other Gram-negative bacteria strains, including two strains of Pseudomonas aeruginosa, two strains of Klebsiella pneumoniae, Acinetobacter baumannii and Stenotrophomonas maltophilia, were also obtained from ATCC.

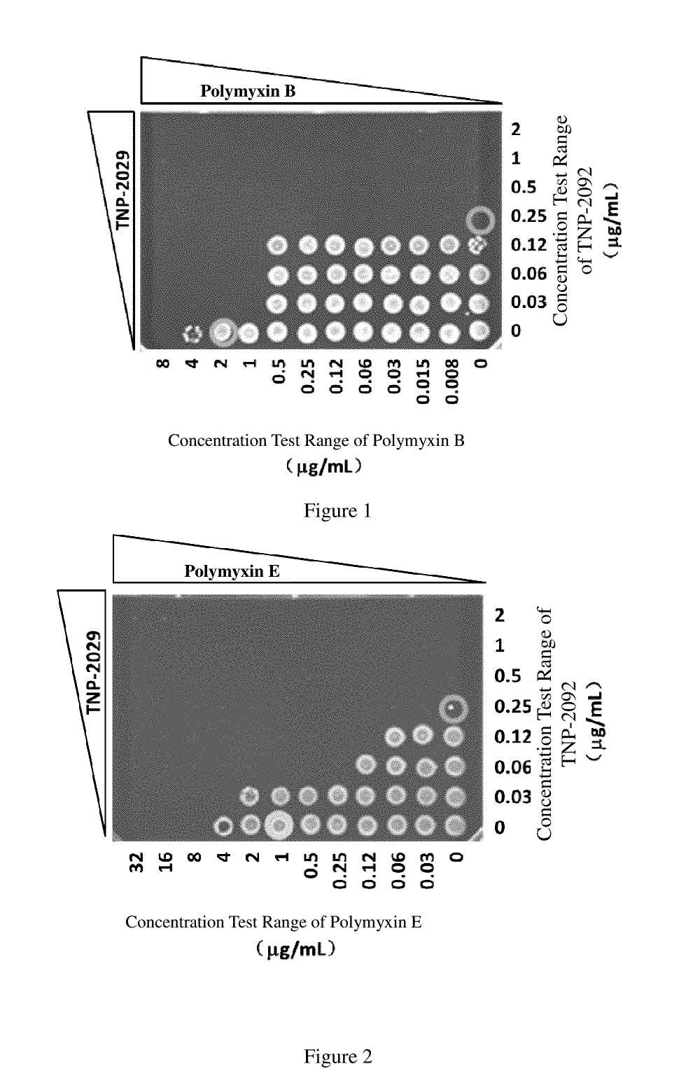

Minimum inhibitory concentration (MIC) endpoints of the combination of TNP-2092 and polymyxin B or E were determined by the two-agent checkerboard assay method based on the CLSI broth microdilution susceptibility method. To obtain a 96-well plate for each pharmaceutical combination, two separate intermediate dilution plates were prepared as described. A sufficient volume of a standard cell inoculum of ˜5×10⁵ CFU/mL was prepared in cation adjusted Mueller Hinton broth with 0.002% (vol:vol) polysorbate-80 using the direct colony suspension method. 0.2 mL of the above suspension was added to the wells in the first column of a first 96-well plate (ID-1), and 0.1 mL of the suspension was added to all remaining wells. 0.2 mL of the same cell inoculum suspension was added to the wells in the first row of a second plate (ID-2), and 0.1 mL of suspension was added to all the remaining wells. An appropriately concentrated drug stock “compound-1” (representing one of the two drugs to be tested in the combination) was added to each well in the first column of the plate ID-1. This was then diluted serially two-fold across the plate, changing tips at each transfer, until column 11 was reached. From column 11, 0.1 mL was removed and discarded and column 12 contains only cell inoculum and no drug. Next, an appropriately concentrated drug stock “Compound 2” (representing the second of the two agents to be tested in the combination) was then diluted into each well of row 1 in plate ID2. This was then diluted serially two-fold down the plate, changing tips at each transfer, until row 7 was reached. 0.1 mL was removed from row 7 and discarded and row 8 contains only cell inoculum and no drug. Finally, 0.05 mL was transferred from both ID1 and ID2 into a third 96 well “destination MIC test plate” (see FIG. 1). This results in a further two fold dilution of the two compounds and yields 77 different test combinations of the two agents. Column 12 is used to call the MIC of compound 2 alone and row 8 is used to call the MIC of compound 1 when tested alone. Note that the intersection of row 8 and column 12 contains no drug and serves as the growth control. The “destination MIC test plates” were then incubated statically at 35° C. for 18-24 h and the MIC of each agent alone or in combination was read visually.

For minimum bactericidal concentration (MBC) endpoints, 0.008 mL portions of each well, after MICs were called, were transferred from the MIC test plates by an automatic sampler to a charcoal agar omni recipient plate. Drops were allowed to air dry in a biological cabinet and the plates incubated inverted for 18-24 h a 35° C. The MBC was scored as the lowest consecutive antimicrobial drug concentration required to kill ≥99.9% of viable input test organisms in 18-24 h, or ≤5 CFU remaining per 0.008 mL drop

In vitro fractional inhibitory concentration (FIC) was calculated using the following formula:

$$FIC = \frac{lowest MIC Agent {X}_{( combination )}}{lowest MIC Agent {X}_{( single agent )}} + \frac{lowest MIC Agent {Y}_{( combination )}}{lowest MIC Agent {Y}_{( single agent )}}$$

FIC (Fractional inhibitory concentration) or FBC (fractional bactericidal concentration) results were interpreted as synergistic, additive, indifferent, or antagonistic. In cases where no endpoint was observed and the MIC/MBC could not be called, then for the purpose of algebraic calculations, the endpoint was arbitrarily assumed to be one dilution greater than the tested range

| FIC or FBC Value | Interpretation |
|---|---|
| ≤0.5 | Synergistic |
| >0.5-1.0 | Additive |
| >1.0-≤4.0 | indifferent |
| >4.0 | Antagonistic |

### Embodiment

Parallel determinations of MIC and MBC endpoints are carried out by checkerboard test of the pharmaceutical combinations. TNP-2092, polymyxin B and polymyxin E have certain antibacterial activity against Escherichia coli ATCC 25922 when tested separately (see Table 1). When the pharmaceutical combination with different proportions of TNP-2092 and polymyxin B or polymyxin E is tested by checkerboard method, if MIC is used as the test endpoint, TNP-2092 demonstrated additive or synergistic effects with polymyxin B or polymyxin E. If MBC is used as the test endpoint, TNP-2092 has a profound synergistic effect with both polymyxin B and polymyxin E (see Table 2, FIG. 1 and FIG. 2). As shown in FIG. 1, the concentration test range for TNP-2092 is 0.03-2 μg/mL, the test range for polymyxin B is 0.008-8 μg/mL. The additive or synergistic effect of TNP-2092 and polymyxin B is observed when the weight ratio is between 0.25:0.008 and 0.03:4. As shown in FIG. 2, the concentration test range for TNP-2092 is 0.03-2 μg/mL, the test range for polymyxin E is 0.03-32 μg/mL, and the additive or synergistic effect of TNP-2092 and polymyxin E is observed when the weight ratio is between 0.25:0.03 and 0.03:4. As a summary, polymyxin B or polymyxin E can enhance the antibacterial activity of TNP-2092 against Escherichia coli and achieve better therapeutic effect.

TABLE 1
MIC, MBC (μg/mL) and MBC_{99.9}/MIC of TNP-2092,
Polymyxin B and Polymyxin E when Used Separately
Against Escherichia coli ATCC 25922

| | | Polymyxin | Polymyxin |
|---|---|---|---|
| Strain | TNP-2092 | B | E |
| Escherichia coli ATCC 25922 (MIC) | 0.25 | 2 | 1 |
| Escherichia coli ATCC 25922 | 0.25 | 8 | 8 |
| (MBC_{99.9}) | | | |
| MBC_{99.9}/MIC | 1 | 4 | 8 |

TABLE 2
FIC and FBC of Pharmaceutical combination of TNP-2092 and Polymyxin
B or Polymyxin E Against Escherichia coli ATCC 25922

| | Cell | | |
|---|---|---|---|
| Compound | permeabilizer | FIC (interpretation) | FBC (interpretation) |
| TNP-2092 | Polymyxin B | 0.564 (Additive) | 0.189 (Synergistic) |
| TNP-2092 | Polymyxin E | 0.500 (Synergistic) | 0.280 (Synergistic) |

For other Gram-negative bacteria, polymyxin B and polymyxin E also have a synergistic effect on the bactericidal activity of TNP-2092, and the results are shown in Table 3 and Table 4. According to the FIC or FBC data obtained by using MIC or MBC as the endpoint, polymyxin B and polymyxin E have additive or synergistic bactericidal effect with TNP-2092. Especially, when MBC is used as the endpoint, TNP-2092 and polymyxin E have a profound synergistic effect (see Table 4).

TABLE 3
FIC of TNP-2092 and Polymyxin B or Polymyxin E
Against a Series of Strains

| | TNP-2029 and | | TNP-2029 | |
|---|---|---|---|---|
| | Polymyxin E | | and Polymyxin B | |
| Strain | FIC | Interpretation | FIC | Interpretation |
| Pseudomonas aeruginosa | 0.504 | Additive | 0.531 | Additive |
| ATCC 10145 | | | | |
| Klebsiella pneumoniae | 0.370 | Synergistic | 0.506 | Additive |
| ATCC 13883 | | | | |
| Stenotrophomonas | 0.516 | Additive | 0.313 | Synergistic |
| maltophilia | | | | |
| ATCC 49130 | | | | |
| Acinetobacter baumannii | 0.280 | Synergistic | 0.280 | Synergistic |
| ATCC 19606 | | | | |
| Escherichia coli | 0.500 | Synergistic | 0.564 | Additive |
| ATCC 25922 | | | | |

TABLE 4
FBC of TNP-2092 and Polymyxin B or
Polymyxin E Against a Series of Strains

| | TNP-2029 | | TNP-2029 | |
|---|---|---|---|---|
| | and Polymyxin E | | and Polymyxin B | |
| Strain | FBC | Conclusion | FBC | Conclusion |
| Pseudomonas aeruginosa | 0.258 | Synergistic | 0.504 | Additive |
| ATCC 10145 | | | | |
| Klebsiella pneumoniae | 0.310 | Synergistic | 0.560 | Additive |
| ATCC 13883 | | | | |
| Klebsiella pneumoniae | 0.254 | Synergistic | 0.750 | Additive |
| ATCC 9997 | | | | |
| Acinetobacter baumannii | 0.266 | Synergistic | 0.127 | Synergistic |
| ATCC 19606 | | | | |
| Escherichia coli | 0.280 | Synergistic | 0.189 | Synergistic |
| ATCC 25922 | | | | |

Therefore, the cell permeabilizer polymyxin B or polymyxin E and TNP-2092 have synergistic antibacterial activity against a series of Gram-negative bacteria. When TNP-2092 is used in combination with polymyxin B or polymyxin E, the inhibitory and bactericidal activities of TNP-2092 against Gram-negative bacteria are significantly enhanced and achieve the goal for treating Gram-negative bacterial infections.

The examples mentioned above are only some embodiments of the present disclosure. For those ordinary skilled in the art, changes and improvements can also be made without departing from the concept of the present disclosure, all of which belong to the scope of the present disclosure.

1. An antibacterial pharmaceutical combination for treating Gram-negative bacteria infections, comprising a cell membrane permeabilizer and a compound TNP-2092 of the following formula:

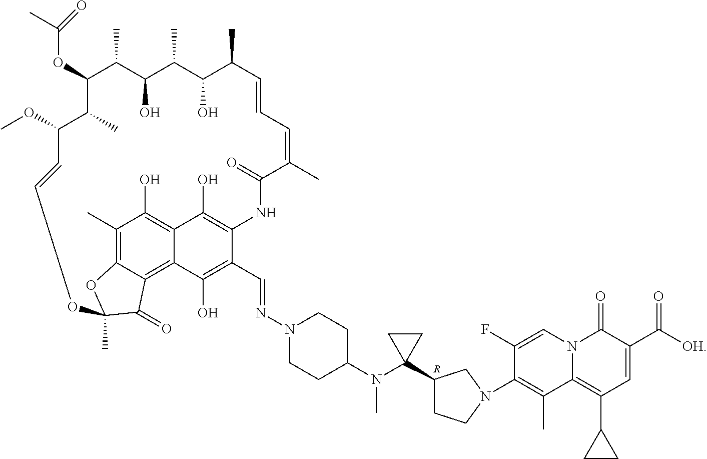

2. The antibacterial pharmaceutical combination for treating Gram-negative bacteria infections according to claim 1, wherein the combination comprises the compound TNP-2092 and the cell membrane permeabilizer with a weight ratio of 3:400-125:4.

3. The antibacterial pharmaceutical combination for treating Gram-negative bacteria infections according to claim 1, wherein the cell membrane permeabilizer is polymyxin B or polymyxin E.

4. The antibacterial pharmaceutical combination for treating Gram-negative bacteria infections according to claim 3, wherein the combination comprises the compound TNP-2092 and polymyxin B with a weight ratio of 3:400-125:4.

5. The antibacterial pharmaceutical combination for treating Gram-negative bacteria infections according to claim 3, wherein the combination comprises the compound TNP-2092 and polymyxin E with a weight ratio of 3:400-25:3.

6. A method for treating Gram-negative bacteria infections in a subject, comprising administrating to the subject the antibacterial pharmaceutical combination of claim 1.

7. The method according to claim 6, wherein the compound TNP-2092 and the cell membrane permeabilizer in the antibacterial pharmaceutical combination are administrated separately, or in a form of a mixture.

8. The method according to claim 6, wherein the Gram-negative bacteria comprises at least one selected from the group consisting of Escherichia coli, Klebsiella pneumoniae, Acinetobacter baumannii, Pseudomonas aeruginosa, Stenotrophomonas maltophilia, Salmonella typhi, non-typhoidal Salmonella and Shigella.
